(19) United States
(12) Patent Application Publication
(10) Pub. No.: US 2006/0263309 A1
(43) Pub. Date: Nov. 23, 2006

(54) Regulation of mammalian keratinous tissue using personal care compositions comprising tetrahydrocurcumin

(76) Inventor: Donald Lynn Bissett, Hamilton, OH (US)

Correspondence Address: THE PROCTER & GAMBLE COMPANY;INTELLECTUAL PROPERTY DIVISION, WINTON HILL BUSINESS CENTER - BOX 161, 6110 CENTER HILL AVENUE, CINCINNATI, OH, 45224, US

(21) Appl. No.: 11/396,738

(22) Filed: Apr. 3, 2006

Related U.S. Application Data
(60) Provisional application No. 60/681,626, filed on May 17, 2005.
(60) Provisional application No. 60/701,370, filed on Jul. 21, 2005.

(51) Int. Cl. A61K 8/97; A61K 8/35

(52) U.S. Cl. 424/59; 424/74

(57) ABSTRACT

Personal care compositions containing tetrahydrocurcumin alone, or in combination with tetrahydromethoxycurcumin and/or tetrahydrobisdemethoxycurcumin are provided. Methods for regulating the condition of mammalian keratinous tissue by topically applying the personal care compositions are also provided.

### CROSS REFERENCE TO RELATED APPLICATION

This application claims the benefit of U.S. Provisional Patent Application Ser. No. 60/681,626, filed May 17, 2005 and U.S. Provisional Patent Application Ser. No. 60/701,370, filed Jul. 21, 2005.

### TECHNICAL FIELD

The present invention relates to personal care compositions containing skin and hair care actives such as tetrahydrocurcumin. Such compositions are useful for regulating the condition of mammalian keratinous tissue needing such treatments, particularly skin lightening.

### BACKGROUND OF THE INVENTION

Currently, there are a number of personal care products that are available to consumers, which are directed toward improving the health and physical appearance of keratinous tissues such as the skin, hair, and nails. The majority of these products are directed to delaying, minimizing or even eliminating skin wrinkling and histological changes typically associated with the aging of skin or environmental damage to human skin. However, there also exists a need for cosmetic agents to prevent, retard, and/or treat uneven skin tone by acting as a lightening or pigmentation reduction cosmetic agent.

Mammalian keratinous tissue, particularly human skin and hair, is subjected to a variety of insults by both extrinsic and intrinsic factors. Such extrinsic factors include ultraviolet radiation, environmental pollution, wind, heat, infrared radiation, low humidity, harsh surfactants, abrasives, etc. Intrinsic factors, on the other hand, include chronological aging and other biochemical changes from within the skin. Whether extrinsic or intrinsic, these factors result in visible signs of skin damage. Typical skin damage includes thinning of the skin, which occurs naturally as one ages. With such thinning, there is a reduction in the cells and blood vessels that supply the skin as well as a flattening of the dermal-epidermal junction that results in weaker mechanical resistance of this junction. See, for example, Oikarinen, “The Aging of Skin: Chronoaging Versus Photoaging,” Photodermatol. Photoimmunol. Photomed., vol. 7, pp. 3-4, 1990. Other damages or changes seen in aging or damaged skin include fine lines, wrinkling, hyperpigmentation, sallowness, sagging, dark under-eye circles, puffy eyes, enlarged pores, diminished rate of turnover, and abnormal desquamation or exfoliation. Additional damage incurred as a result of both external and internal factors includes visible dead skin (i.e., flaking, scaling, dryness, roughness). For hair, these extrinsic and intrinsic factors can contribute to, among other problems, hair bleaching, split ends, fragility, roughness, hair loss, reduction in hair growth rate, and the like. Therefore, there is a need for products and methods that seek to remedy these keratinous tissue conditions.

### SUMMARY OF THE INVENTION

Applicants have discovered that topical compositions that contain certain actives may be used to provide prophylactic as well as therapeutic treatments for keratinous tissue conditions, particularly skin-lightening.

In accordance with one preferred embodiment, there has now been provided a personal care composition comprising tetrahydrocurcumin; a safe and effective amount of at least one additional skin and/or hair care active selected from the group consisting of sugar amine, vitamin B₃, retinoids, hydroquinone, peptides, phytosterol, dialkanoyl hydroxyproline, hexamidine, salicylic acid, n-acyl amino acid compounds, sunscreen actives, water soluble vitamins, oil soluble vitamins, hesperedin, mustard seed extract, glycyrrhizic acid, glycyrrhetinic acid, carnosine, Butylated Hydroxytoluene (BHT) and Butylated Hydroxyanisole (BHA), menthyl anthranilate, cetyl pyridinium chloride, ergothioneine, vanillin or its derivatives, diethylhexyl syrinylidene malonate, melanostatine, sterol esters, dehydroacetic acid, idebenone, yeast extract, beta glucans, alpha glucans, their salts, their derivatives, their precursors, and/or combinations thereof; and a dermatologically acceptable carrier.

In accordance with another preferred embodiment, there has now been provided a personal care composition comprising at least one anti-oxidant compound, at least one tyrosinase inhibiting compound, at least one trypsin inhibiting compound, at least one anti-inflammatory compound, and at least one nitric oxide scavenging compound.

In accordance with yet another preferred embodiment, there has now been provided an article of commerce comprising a personal care composition; and at least one of the personal care composition, packaging for the personal care composition and advertisement material pertaining to the personal care composition comprising indicia and/or an image which communicates that topical application of the personal care compostion may improve one's skin tone or skin color. The personal care composition includes tetrahydrocurcumin and at least one sunscreen active.

The invention further relates to methods for regulating the condition of mammalian keratinous tissue wherein the methods each comprise the step of topically applying to the keratinous tissue of a mammal needing such treatment, a safe and effective amount of a personal care composition in accordance with the invention.

### DETAILED DESCRIPTION OF THE INVENTION

All percentages and ratios used herein are by weight of the total composition and all measurements made are at 25° C., unless otherwise designated.

The compositions of the present invention can comprise, consist essentially of, or consist of, the essential components as well as optional ingredients described herein. As used herein, “consisting essentially of” means that the composition or component may include additional ingredients, but only if the additional ingredients do not materially alter the basic and novel characteristics of the claimed compositions or methods.

The term “keratinous tissue,” as used herein, refers to keratin-containing layers disposed as the outermost protective covering of mammals which includes, but is not limited to, skin, hair, toenails, fingernails, cuticles, hooves, etc.

The term “topical application,” as used herein, means to apply or spread the compositions of the present invention onto the surface of the keratinous tissue.

The term “dermatologically acceptable,” as used herein, means that the compositions or components described are suitable for use in contact with human keratinous tissue without undue toxicity, incompatibility, instability, allergic response, and the like.

The term “safe and effective amount,” as used herein, means an amount of a compound or composition sufficient to significantly induce a positive benefit, preferably a positive keratinous tissue appearance or feel benefit, including independently or in combination the benefits disclosed herein, but low enough to avoid serious side effects (i.e., to provide a reasonable benefit to risk ratio, within the scope of sound judgment of the skilled artisan).

The term “post-inflammatory hyperpigmentation,” as used herein, refers to the changes in melanin content as a response to an inflammatory event (e.g., acne, scratch, insect sting or bite, sunburn, etc), especially in dark skin subjects.

The term “hyperpigmentation,” as used herein, refers to an area of skin wherein the pigmentation is greater than that of an adjacent area of skin (e.g., a pigment spot, an age spot, and the like).

The terms “desquamation, exfoliation, and/or turnover,” as used herein, mean the removal of the upper layers of the stratum corneum (comprising the horny layers).

The terms “oily and/or shiny appearance,” as used herein, mean the glossy look mammalian skin tends to exhibit upon the excretion of oil, sebum, and/or sweat from the respective source gland.

The term “sagging,” as used herein means the laxity, slackness, or the like condition of skin that occurs as a result of loss of, damage to, alterations to, and/or abnormalities in dermal elastin.

The terms “smoothing” and “softening,” as used herein, mean altering the surface of the keratinous tissue such that its tactile feel is improved.

The term “sallowness,” as used herein, means the pale color, yellow color or the like condition of skin that occurs as a result of a loss of, damage to, alterations to, and/or abnormalities in skin components such that they become colored (e.g., yellow in color) due to processes such as protein glycation and accumulation of lipofuscin or in the decrease in peripheral blood flow that typically accompanies skin aging.

The compositions of the present invention are useful for topical application and for regulating keratinous tissue condition. Regulation of keratinous tissue condition, especially human skin condition, is often required due to conditions that may be induced or caused by factors internal and/or external to the body. For instance, “regulating skin condition” includes prophylactically regulating and/or therapeutically regulating skin condition, and may involve one or more of the following benefits: thickening (i.e., building the epidermis and/or dermis layers of the skin and/or the subcutaeous layers such as fat and muscle and where applicable the keratinous layers of the nail and hair shaft) to reduce atrophy (e.g., of the skin); increasing the convolution of the dermal-epidermal border; decreasing non-melanin skin discoloration such as under eye circles, blotching (e.g., uneven red coloration due to, e.g., rosacea) (hereinafter referred to as “red blotchiness”), and sallowness (pale or yellow color), decreasing discoloration caused by telangiectasia or spider vessels, decreasing discolorations due to melanin (e.g., pigment spots, age spots, uneven pigmentation) and other chromophores in the skin (e.g., lipofuscin, protein crosslinks such as those that occur with glycation, and the like). As used herein, prophylactically regulating skin condition includes delaying, minimizing and/or preventing visible and/or tactile discontinuities in skin (e.g., texture irregularities, fine lines, wrinkles, sagging, stretch marks, cellulite, puffy eyes, and the like in the skin which may be detected visually or by feel). As used herein, therapeutically regulating skin condition includes ameliorating (e.g., diminishing, minimizing and/or effacing) discontinuities in skin. Regulating skin condition involves improving skin appearance and/or feel.

The term “regulating skin condition,” as used herein, is intended to include regulation of such signs irrespective of the mechanism of origin.

The term “indicia,” as used herein means an identifying mark, including text and/or graphics.

The term “image,” as used herein means a photograph, illustration and/or other pictorial representation of a mammal or object.

The term “packaging,” as used herein means a structure or material that is at least partially disposed on or about a personal care composition when the product is presented to the public. “Primary packaging” means any container, including its closure, pump, cap or other peripheral items, in which the composition is in direct contact. And “secondary packaging” means any additional materials that are associated with the primary packaging, such as, for example, a container such as a box or polymeric sleeve that at least partially surrounds, contains, or contacts the primary packaging.

The term “advertisement material,” as used herin means a tangible medium of expression, which by itself or with the aid of a peripheral device, makes known the existence of, or proclaims the quality or advantages of, an associated personal care composition.

The compositions of the present invention, including the essential and optional components thereof, are described in detail hereinafter.

### Components

Tetrahydrocurcumin

The present invention comprises a safe and effective amount of tetrahydrocurcumin (THC), its derivatives, such as esters or ethers, and combinations thereof. Preferably, the composition contains the tetrahydrocurcumin compound from about 0.01% to about 10%, more preferably from about 0.1% to about 5%, even more preferably from about 0.25% to about 3%, by weight of the composition.

Oxygen radicals are produced in the skin in response to many stimuli, such as exposure to UV and irritants. Such radicals are also produced as by-products of normal cell or tissue metabolism. Oxygen radicals can stimulate pigment cells (melanocytes) to increase production of melanin. Curcuminoids (such as THC) have anti-oxidant properties and thus can scavenge oxygen radicals before they stimulate the melanocytes.

The protein tyrosinase is an enzyme involved in the conversion of the amino acid tyrosine to DOPA (dihydroxyphenylalanine) which then is further converted into other intermediates and polymerized into the skin pigment melanin. Partial or complete inhibition of tyrosinase slows or stops, respectively, the formation of melanin, leading to lighter skin color (e.g., reduction in darkness of hyperpigmented spots). Curcuminoids (such as THC) also inhibit tyrosinase.

Other curcuminoids useful in the present invention include curcumin, tetrahydrodemethoxycurcumin, tetrahydrobisdemethoxycurcumin, their derivatives such as esters and ethers, and combinations thereof. Curcuminoids (such as THC) can be of natural or synthetic origin. Preferably, in the present invention, tetrahydrocurcumin is used alone or in combination with tetrahydrodemethoxycurcumin and/or in combination with tetrahydrobisdemethoxycurcumin. Preferred derivatives are esters, particularly the diacetate ester of tetrahydrocurcumin and/or its combination with tetrahydrocurcumin and/or its combination with tetrahydrodemethoxycurcumin and/or its combination with tetrahydrobisdemethoxycurcumin and/or its combination with the esters, particularly the diacetate ester, of tetrahydrodemethoxycurcumin and/or the diacetate ester of tetrahydrobisdemethoxycurcumin.

Additional Skin and/or Hair Care Actives

The present invention comprises a safe and effective amount of at least one additional skin and/or hair care active. These skin and/or hair care actives are selected from the group consisting of sugar amine, vitamin B₃, retinoids, hydroquinone, peptides, phytosterol, dialkanoyl hydroxyproline, hexamidine, salicylic acid, n-acyl amino acid compounds, sunscreen actives, water soluble vitamins, oil soluble vitamins, hesperedin, mustard seed extract, glycyrrhizic acid, glycyrrhetinic acid, camosine, Butylated Hydroxytoluene (BHT) and Butylated Hydroxyanisole (BHA), menthyl anthranilate, cetyl pyridinium chloride, ergothioneine, vanillin or its derivatives, diethylhexyl syrinylidene malonate, melanostatine, sterol esters, idebenone, dehydroacetic acid, yeast extract (e.g., Pitera®), beta glucans, alpha glucans, their salts, their derivatives, their precursors, and/or combinations thereof. Further description of some of these additional actives is provided below.

The skin and/or hair care actives of the present invention may be useful in skin lightening. Skin lightening may occur through multiple mechanisms including anti-oxidant mechanisms, trypsin inhibition, anti-inflammatory mechanisms, nitric oxide scavenging, tyrosinase inhibition, etc. Thus, compounds which have these mechanisms have the potential to lighten skin.

1. Sugar Amines (Amino Sugars)

The compositions of the present invention optionally include a safe and effective amount of a sugar amine, which are also known as amino sugars. The sugar amine compounds useful in the present invention are described in PCT Publication WO 02/076423 and U.S. Pat. No. 6,159,485.

Preferably, the composition contains from about 0.01% to about 15%, more preferably from about 0.1% to about 10%, and even more preferably from about 0.5% to about 5% by weight of the composition, of the sugar amine.

Sugar amines can be synthetic or natural in origin and can be used as pure compounds or mixtures of compounds (e.g., extracts from natural sources or mixtures of synthetic materials). Glucosamine is generally found in many shellfish and can also be derived from fungal sources. As used herein, “sugar amine” includes isomers and tautomers of such and its salts (e.g., HCl salt) and is commercially available from Sigma Chemical Co.

Examples of sugar amines that are useful herein include glucosamine, N-acetyl glucosamine, glucosamine sulfate, mannosamine, N-acetyl mannosamine, galactosamine, N-acetyl galactosamine, their isomers (e.g., stereoisomers), and their salts (e.g., HCl salt). Preferred for use herein are glucosamine, particularly D-glucosamine and N-acetyl glucosamine, particularly N-acetyl-D-glucosamine.

2. Vitamin B₃

The compositions of the present invention may include a safe and effective amount of a vitamin B₃ compound. Vitamin B₃ compounds are particularly useful for regulating skin condition as described in U.S. Pat. No. 5,939,082. Preferably, the composition contains from about 0.01% to about 50%, more preferably from about 0.1% to about 20%, even more preferably from about 0.5% to about 10%, and still more preferably from about 1% to about 7%, even more preferably from about 2% to about 5%, by weight of the composition, of the vitamin B₃ compound.

As used herein, “vitamin B₃ compound” means a compound having the formula: 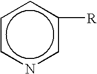
wherein R is —CONH₂ (i.e., niacinamide), —COOH (i.e., nicotinic acid) or —CH₂OH (i.e., nicotinyl alcohol); derivatives thereof; and salts of any of the foregoing.

Exemplary derivatives of the foregoing vitamin B₃ compounds include nicotinic acid esters, including non-vasodilating esters of nicotinic acid (e.g., tocopheryl nicotinate, myristyl nicotinate).

Examples of suitable vitamin B₃ compounds are well known in the art and are commercially available from a number of sources (e.g., the Sigma Chemical Company, ICN Biomedicals, Inc., and Aldrich Chemical Company). A preferred vitamin B₃ compound useful in the present invention is niacinamide.

3. Retinoids

The compositions of this invention may contain a safe and effective amount of a retinoid, such that the resultant composition is safe and effective for regulating keratinous tissue condition, preferably for regulating visible and/or tactile discontinuities in skin, more preferably for regulating signs of skin aging. The compositions preferably contain from about 0.001% to about 10%, more preferably from about 0.005% to about 2%, even more preferably from about 0.01% to about 1%, still more preferably from about 0.01% to about 0.5%, by weight of the composition, of the retinoid. The optimum concentration used in a composition will depend on the specific retinoid selected since their potency does vary considerably.

As used herein, “retinoid” includes all natural and/or synthetic analogs of Vitamin A or retinol-like compounds which possess the biological activity of Vitamin A in the skin as well as the geometric isomers and stereoisomers of these compounds. The retinoid is preferably selected from retinol, retinol esters (e.g., C₂-C₂₂ alkyl esters of retinol, including retinyl palmitate, retinyl acetate, retinyl propionate), retinal, and/or retinoic acid (including all-trans retinoic acid and/or 13-cis-retinoic acid), or mixtures thereof. More preferably the retinoid is a retinoid other than retinoic acid. Preferred retinoids are retinol, retinyl palmitate, retinyl acetate, retinyl propionate, retinal and combinations thereof. More preferred is retinyl propionate, used even more preferably from about 0.1% to about 0.3%.

4. Peptides

The compositions of the present invention may contain a safe and effective amount of a peptide, including but not limited to, di-, tri-, tetra-, penta-, and hexa-peptides and derivatives thereof. The compositions contain preferably from about 1×10⁻⁶% to about 20%, more preferably from about 1×10⁻⁶% to about 10%, even more preferably from about 1×10⁻⁵% to about 5%, by weight of the composition.

As used herein, “peptide” refers to peptides containing ten or fewer amino acids and their derivatives, isomers, and complexes with other species such as metal ions (e.g., copper, zinc, manganese, magnesium, and the like). As used herein, peptide refers to both naturally occurring and synthesized peptides. Also useful herein are naturally occurring and commercially available compositions that contain peptides. More preferred peptides are the dipeptide carnosine (beta-ala-his), the tripeptide gly-his-lys, the pentapeptide lys-thr-thr-lys-ser, lipophilic derivatives of peptides, and metal complexes of the above, e.g., copper complex of the tripeptide his-gly-gly (also known as Iamin). A preferred dipeptide derivative is palmitoyl-lys-thr. A preferred commercially available tripeptide derivative-containing composition is Biopeptide CL®, which contains 100 ppm of palmitoyl-gly-his-lys and is commercially available from Sederma. A preferred commercially available pentapeptide derivative-containing composition is Matrixyl®, which contains 100 ppm of palmitoyl-lys-thr-thr-lys-ser and is commercially available from Sederma.

5. Phytosterol

The topical compositions of the present invention may comprise a safe and effective amount of one or more phytosterols selected from the group consisting of β-sitosterol, campesterol, brassicasterol, Δ5-avennasterol, lupenol, α-spinasterol, stigmasterol, their derivatives, analogs, and combinations thereof. More preferably, the phytosterol is selected from the group consisting of β-sitosterol, campesterol, brassicasterol, stigmasterol, their derivatives, and combinations thereof. More preferably, the phytosterol is stigmasterol.

Phytosterols can be synthetic or natural in origin and can be used as essentially pure compounds or mixtures of compounds (e.g., extracts from natural sources). Phytosterols are generally found in the unsaponifiable portion of vegetable oils and fats and are available as free sterols, acetylated derivatives, sterol esters, ethoxylated or glycosidic derivatives. More preferably, the phytosterols are free sterols. As used herein, “phytosterol” includes isomers and tautomers of such and is commercially available from Aldrich Chemical Company, Sigma Chemical Company, and Cognis.

In the compositions of the present invention, the phytosterol preferably comprises from about 0.0001% to about 25%, more preferably from about 0.001% to about 15%, even more preferably from about 0.01% to about 10%, still more preferably from about 0.1% to about 5%, and even more preferably from about 0.2% to about 2% by weight of the composition.

6. Hexamidine

The hexamidine compounds useful in the present invention correspond to those of the following chemical structure: 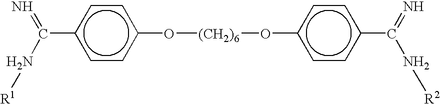
wherein R¹ and R² comprise organic acids (e.g., sulfonic acids, etc.).

In the composition of the present invention, the hexamidine preferably comprises from about 0.0001 to about 25%, more preferably from about 0.001 to about 10%, more preferably from about 0.01 to about 5%, and even more preferably from about 0.02 to about 2.5% by weight of the composition.

The topical compositions of the present invention optionally include a safe and effective amount of one or more of hexamidine compounds, its salts, and its derivatives. As used herein, hexamidine derivatives include any isomers and tautomers of hexamidine compounds including but not limited to organic acids and mineral acids, for example sulfonic acid, carboxylic acid etc. Preferably, the hexamidine compounds include hexamidine diisethionate, commercially available as Eleastab® HP100 from Laboratoires Serobiologiques.

7. Dialkanoyl Hydroxyproline Compounds

The dialkanoyl hydroxyproline compounds of the present invention correspond to those of the following chemical structure: 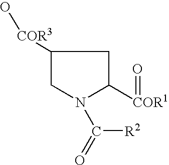
wherein R¹ comprises H, X, C₁-C₂₀ straight or branched alkyl,

X comprises metals (Na, K, Li, Mg, Ca) or amines (DEA, TEA);

R² comprises C₁-C₂₀ straight or branched alkyl;

R³ comprises C₁-C₂₀ straight or branched alkyl.

The topical compositions of the present invention may comprise a safe and effective amount of one or more dialkanoyl hydroxyproline compounds and their salts and derivatives. In the composition of the present invention, the dialkanoyl hydroxyproline compounds preferably comprise from about 0.01 to 10%, more preferably from about 0.1-5%, even more preferably from about 0.1 to 2% by weight of the composition

Suitable derivatives include but are not limited to esters, for example fatty esters, including, but not limited to tripalmitoyl hydroxyproline and dipalmityl acetyl hydroxyproline. A particularly useful compound is dipalmitoyl hydroxyproline. As used herein, dipalmitoyl hydroxyproline includes any isomers and tautomers of such and is commercially available under the tradename Sepilift DPHP® from Seppic, Inc. Further discussion of dipalmitoyl hydroxyproline appears in PCT Publication WO 93/23028. Preferably the dipalmitoyl hydroxyproline is the triethanolamine salt of dipalmitoyl hydroxyproline.

8. Salicylic Acid Compounds

The topical compositions of the present invention may comprise a safe and effective amount of a salicylic acid compound, its esters, its salts, or combinations thereof. In the compositions of the present invention, the salicylic acid compound preferably comprises from about 0.0001% to about 25%, more preferably from about 0.001% to about 15%, even more preferably from about 0.01% to about 10%, still more preferably from about 0.1% to about 5%, and even more preferably from about 0.2% to about 2%, by weight of the composition, of salicylic acid.

9. N-acyl Amino Acid Compounds

The topical compositions of the present invention may comprise a safe and effective amount of one or more N-acyl amino acid compounds. The amino acid can be one of any of the amino acids known in the art. The N-acyl amino acid compounds of the present invention correspond to the formula: 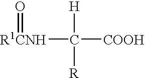
wherein R can be a hydrogen, alkyl (substituted or unsubstituted, branched or straight chain), or a combination of alkyl and aromatic groups. A list of possible side chains of amino acids known in the art are described in Stryer, Biochemistry, 1981, published by W. H. Freeman and Company. R¹ can be C₁ to C₃₀, saturated or unsaturated, straight or branched, substituted or unsubstituted alkyls; substituted or unsubstituted aromatic groups; or mixtures thereof.

Preferably, the N-acyl amino acid compound is selected from the group consisting of N-acyl Phenylalanine, N-acyl Tyrosine, their isomers, their salts, and derivatives thereof. The amino acid can be the D or L isomer or a mixture thereof. N-acyl Phenylalanine corresponds to the following formula: 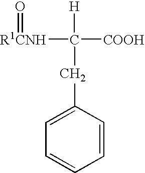
wherein R¹ can be C₁ to C₃₀, saturated or unsaturated, straight or branched, substituted or unsubstituted alkyls; substituted or unsubstituted aromatic groups; or mixtures thereof.

N-acyl Tyrosine corresponds to the following formula: 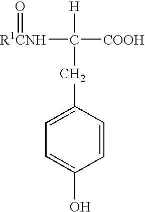
wherein R¹ can be C₁ to C₃₀, saturated or unsaturated, straight or branched, substituted or unsubstituted alkyls; substituted or unsubstituted aromatic groups; or mixtures thereof.

Particularly useful as a topical skin tone evening (lightening or pigmentation reduction) cosmetic agent is N-undecylenoyl-L-phenylalanine. This agent belongs to the broad class of N-acyl Phenylalanine derivatives, with its acyl group being a C11 mono-unsaturated fatty acid moiety and the amino acid being the L-isomer of phenylalanine. N-undecylenoyl-L-phenylalanine corresponds to the following formula:

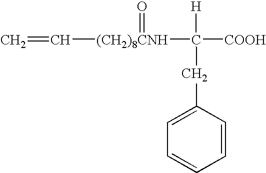

As used herein, N-undecylenoyl-L-phenylalanine is commercially available under the tradename Sepiwhite® from SEPPIC.

In the composition of the present invention, the N-acyl amino acid preferably comprises from about 0.0001-25%, more preferably from about 0.001-10%, more preferably from about 0.01-5%, and even more preferably from about 0.02-2.5% by weight of the composition.

10. Sunscreen Actives

The compositions of the subject invention may optionally contain a sunscreen active. As used herein, “sunscreen active” includes both sunscreen agents and physical sunblocks. Suitable sunscreen actives may be organic or inorganic.

A wide variety of conventional sunscreen actives are suitable for use herein. Sagarin, et al., at Chapter VIII, pages 189 et seq., of Cosmetics Science and Technology (1972), discloses numerous suitable actives. Particularly suitable sunscreen agents are 2-ethylhexyl-p-methoxycinnamate (commercially available as PARSOL MCX), 4,4′-t-butyl methoxydibenzoyl-methane (commercially available as PARSOL 1789), 2-hydroxy-4-methoxybenzophenone, octyldimethyl-p-aminobenzoic acid, digalloyltrioleate, 2,2-dihydroxy-4-methoxybenzophenone, ethyl-4-(bis(hydroxy-propyl))aminobenzoate, 2-ethylhexyl-2-cyano-3,3-diphenylacrylate, 2-ethylhexyl-salicylate, glyceryl-p-aminobenzoate, 3,3,5-tri-methylcyclohexylsalicylate, methylanthranilate, p-dimethyl-aminobenzoic acid or aminobenzoate, 2-ethylhexyl-p-dimethyl-amino-benzoate, 2-phenylbenzimidazole-5-sulfonic acid, 2-(p-dimethylaminophenyl)-5-sulfonicbenzoxazoic acid, octocrylene, zinc oxide, titanium dioxide, and mixtures of these compounds.

Preferred organic sunscreen actives useful in the compositions of the present invention are 2-ethylhexyl-p-methoxycinnamate, butylmethoxydibenzoyl-methane, 2-hydroxy-4-methoxybenzo-phenone, 2-phenylbenzimidazole-5-sulfonic acid, octyldimethyl-p-aminobenzoic acid, octocrylene, zinc oxide, titanium dioxide, and mixtures thereof. Especially preferred sunscreen actives include 4,4′-t-butylmethoxydibenzoylmethane, 2-ethylhexyl-p-methoxycinnamate, phenyl benzimidazole sulfonic acid, octocrylene, zinc oxide, and titanium dioxide, and mixtures thereof.

The sunscreen active preferably comprises from about 1% to about 20%, more preferably from about 2% to about 10%, by weight of the composition. Exact amounts will vary depending upon the sunscreen chosen and the desired Sun Protection Factor (SPF).

11. Water-Soluble Vitamins

The compositions of the present invention may contain a safe and effective amount of one or more water-soluble vitamins. Examples of water-soluble vitamins include, but are not limited to, water-soluble versions of vitamin B (such as vitamin B5 and vitamin B6 (e.g., pyridoxine)), vitamin B derivatives, vitamin C (such as ascorbyl glucoside), vitamin C derivatives (such as magnesium ascorbyl phosphate, sodium ascorbyl phosphate, and ascorbyl palmitate), vitamin K, vitamin K derivatives, pro-vitamins thereof, such as panthenol and mixtures thereof. When vitamin compounds are present in the compositions of the instant invention, the compositions preferably contain from about 0.0001% to about 50%, more preferably from about 0.001% to about 10%, still more preferably from about 0.01% to about 8%, and still more preferably from about 0.1% to about 5%, by weight of the composition, of the vitamin compound.

12. Oil-Soluble Vitamins

The compositions of the present invention may contain a safe and effective amount of one or more oil-soluble vitamins. Examples of oil-soluble vitamins include, but are not limited to, oil-soluble versions of vitamin D, vitamin D derivatives, vitamin E (such as vitamin E acetate), vitamin E derivatives, pro-vitamins thereof, and mixtures thereof. When oil-soluble vitamin compounds are present in the compositions of the instant invention, the compositions preferably contain from about 0.0001% to about 50%, more preferably from about 0.001% to about 10%, still more preferably from about 0.01% to about 8%, and still more preferably from about 0.1% to about 5%, by weight of the composition, of the oil-soluble vitamin compound.

13. Hesperedin

The compositions of the present invention may include a safe and effective amount of hesperedin. The hesperdin of the composition may preferably be its derivative glucosyl herperidin. Preferably, the composition contains from about 0.01% to about 10%, more preferably from about 0.1% to about 5%, even more preferably from about 0.5% to about 3%, by weight of the composition, of the hesperedin compound.

Hesperidin and glucosyl hesperedin are flavonoids. Oxygen radicals are produced in the skin in response to many stimuli, such as exposure to UV and irritatants. Such radicals are also produced as by-products of normal cell or tissue metabolism. Oxygen radicals can stimulate pigment cells (melanocytes) to increase production of melanin. Hesperidin and glucosyl hesperedin have anti-oxidant properties and thus can scavenge oxygen radicals before they stimulate the melanocytes.

The protein tyrosinase is an enzyme involved in the conversion of the amino acid tyrosine to DOPA (dihydroxyphenylalanine) which then is further converted into other intermediates and polymerized into the skin pigment melanin. Partial or complete inhibition of tyrosinase slows or stops, respectively, the formation of melanin, leading to lighter skin color (e.g., reduction in darkness of hyperpigmented spots). Hesperidin and glucosyl hesperidin also inhibit tyrosinase.

14. Glycyrrhizic acid

The compositions of the present invention may include a safe and effective amount of glycyrrhizic acid and/or its salts. Preferably, the composition contains from about 0.01% to about 10%, more preferably from about 0.05% to about 5%, even more preferably from about 0.1% to about 3%, by weight of the composition, of the glycyrrhizic acid compound. Glycyrrhizic acid is a component of licorice extract.

Glycyrrhizic acid is an anti-inflammatory agent. Inflammatory mediators or cytokines can stimulate pigment cells (melanocytes) to produce melanin. Thus inflammatory conditions such as UV-damage, acne, in-grown hairs, insect bites, scratches, etc. will stimulate what is called post-inflammatory hyperpigmentation. While UV is a primary inducer of pigmentation in all skin types, pigment from the other inflammatory stimuli (acne, etc.) will in particular contribute to skin pigmentation in darker skin individuals (e.g., Hispanic, Asian). Inhibiting inflammation with anti-inflammatory agents will reduce pigmentation.

Glycyrrhizic acid is also believed to be a scavenger of nitric oxide. Nitric oxide (NO) is a stimulator of pigmentation. Use of nitric oxide scavengers (materials that react with nitric oxide to prevent it from stimulating pigment cells) will reduce pigmentation.

Glycyrrhizic acid is also known as glycyrrhizin, glycyrrhizinic acid, or glycyrrhetinic acid glycoside.

15. Glycyrrhetinic Acid

The compositions of the present invention may include a safe and effective amount of glycyrrhetinic acid and/or its salts. Preferably, the composition contains from about 0.01% to about 10%, more preferably from about 0.05% to about 5%, even more preferably from about 0.1% to about 3%, by weight of the composition, of the glycyrrhetinic acid compound. Glycyrrhetinic acid is a component of licorice extract.

Glycyrrhetinic acid is also an anti-inflammatory agent, discussed above in the glycyrrhizic acid section. Structurally, glycyrrhetinic acid is different from glycyrrhizic acid in that glycyrrhetinic acid does not have an attached sugar residue (glycoside). Glycyrrhetinic acid is also known as enoxolone, glycyrrhetic acid, or uralenic acid.

16. Carnosine

The compositions of the present invention may include a safe and effective amount of carnosine. Preferably, the composition contains from about 0.01% to about 20%, more preferably from about 0.1% to about 15%, even more preferably from about 1% to about 10%, by weight of the composition, of the camosine compound.

Carnosine is a dipeptide and acts as an anti-oxidant. The anti-oxidant mechanism is the same as that described above in hesperidin section. Carnosine is found naturally in the human body. It has been called the anti-aging peptide since it is present in high levels in longer-lived tissues and is present at low levels in tissues with issues (e.g., cataracts). Materials that are structurally and mechanistically similar to carnosine include carcinine, anserrine, homocarnosine and ophidine.

17. Cetyl Pyridinium Chloride

The compositions of the present invention may comprise a safe and effective amount of cetyl pyridinium chloride. Alternate forms of cetyl pyridinium chloride include those in which one or two of the substitutes on the quaternary nitrogen has a carbon chain length (typically alkyl group) from about 8 to about 20, typically from about 10 to about 18 carbon atoms while the remaining substitutes (typically alkyl or benzyl group) have a lower number of carbon atoms, such as from about 1 to about 7 carbon atoms (typically methyl or ethyl groups). Dodecyl trimethyl ammonium bromide, tetradecylpyridinium chloride, domiphenbromide, N-tetradecyl-4-ethyl pyridinium chloride, dodecyl dimethyl (2-phenoxyethyl) ammonium bromide, benzyl dimethylstearyl ammonium chloride, quaternized 5-amino-1,3-bis(2-ethyl-hexyl)-5-methyl hexahydropyrimidine, benzalkonium chloride, benzethonium chloride and methyl benzethonium chloride are exemplary of typical quaternary ammonium agents. Other compounds are bis-4-(R-amino)-1-pyridinium alkanes as disclosed in U.S. Pat. No. 4,206,215.

Cetyl pyridinium chloride may be present in an amount of from about 0.005% to about 10% by weight of the composition, more preferably from about 0.01% to about 5%, more preferably from about 0.05% to about 2%. Cetyl pyridinium chloride is an inhibitor of tyrosinase, a mechanism discussed above.

18. Ergothioneine

The compositions of the present invention may comprise a safe and effective amount of ergothioneine. Ergothioneine may be present in an amount of from about 0.01% to about 20% by weight of the composition, more preferably from about 0.1% to about 15% by weight of the composition, even more preferably from about 1% to about 10% by weight of the composition. A preferred ergothioneine is Thiotaine® which is a commercial solution of the chemical ergothioneine, commercially available from Bamet Products. Ergothioneine exhibits anti-oxidant properties, a mechanism described above.

19. Diethylhexyl Syrinylidene Malonate

The compositions of the present invention may comprise a safe and effective amount of diethylhexyl syrinylidene malonate. Diethylhexyl syrinylidene malonate may be present in an amount of from about 0.01% to about 20% by weight of the composition, more preferably from about 0.1% to about 15% by weight of the composition, even more preferably from about 0.5% to about 10% by weight of the composition.

A preferred diethylhexyl syrinylidene malonate is Oxynex® which exhibits anti-oxidant properties. It is available from Rona/Merck.

20. Melanostatine

The compositions of the present invention may comprise a safe and effective amount of melanostatine. Melanostatine may be present in an amount of from about 0.01% to about 20% by weight of the composition, more preferably from about 0.1% to about 15% by weight of the composition, even more preferably from about 0.5% to about 10% by weight of the composition. Since melanostatine is a commercial solution of peptide (approximately 50 ppm peptide in this commercial solution), the actual level of peptide in a product containing 5% melanostatine actually contains approximately 2.5 ppm peptide).

A preferred melanostatine is available from Vincience (France). Melanostatine is a hexapeptide, and it operates mechanistically by inhibiting binding of alpha-MSH (melanin stimulating hormone) to its cell receptor, thus inhibiting initiation of pigmentation.

21. Sterol Esters

The compositions of the present invention may comprise a safe and effective amount of sterol esters. The sterol esters may be present in an amount of from about 0.01% to about 20% by weight of the composition, more preferably from about 0.1% to about 15% by weight of the composition, even more preferably from about 0.5% to about 10% by weight of the composition.

When sterol esters are used in the present invention, formulation of the composition should be performed so that hydrolysis of the esters does not occur. Therefore, the ideal pH range of the composition comprising sterol esters is from about 3 to about 8, preferably from about 4 to about 7.

Sterol esters useful in the present invention may be comprised of sterols or mixtures of sterols (in particular sitosterol, campesterol, stigmasterol, brassicasterol, and additional sterols) which are esterified with a fatty acid or mixtures of fatty acids (which can be straight chain or branched chain, saturated or unsatured) with from 8 to 30 carbon atoms (preferably 16-22 carbon atoms). Sterol esters are available from P&G Chemicals.

22. Idebenone

The compositions of the present invention may include a safe and effective amount of idebenone. Preferably, the composition contains from about 0.001% to about 10%, more preferably from about 0.005% to about 5%, even more preferably from about 0.01% to about 1% by weight of the composition, of the idebenone compound. Alternatively, the composition may comprise from about 0.1% to about 0.5% by weight of the composition of the idebenone compound.

Herein, “idebenone” means the following compound, its esters and other derivatives, salts, isomers, tautomers, and combinations thereof:

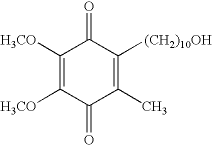

One technical name for idebenone of the present invention is 6-(10-hydroxydecyl)-2,3 ,-dimethoxy-5-methyl-1,4-benzoquinone.

23. Dehydroacetic Acid

The composition of the present invention may include dehydroacetic acid, having the structure: 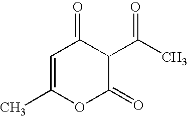
or pharmaceutically acceptable salts, derivatives or tautomers thereof. As used herein, “pharmaceutically acceptable” means that the salts of dehydroacetic acid are suitable for use in contact with the tissues of mammals to which they will be exposed without undue toxicity, incompatibility, instability, irritation, allergic response, and the like. The technical name for dehydroacetic acid is 3-Acetyl-6-methyl-2H-pyran-2,4(3H)-dione and can be commercially purchased from Lonza.

Pharmaceutically acceptable salts include alkali metal salts, such as sodium and potassium; alkaline earth metal salts, such as calcium and magnesium; non-toxic heavy metal salts; ammonium salts; and trialkylammonium salts, such astrimethylammonium and triethylammonium. Sodium, potassium, and ammonium salts of dehydroacetic acid are preferred. Highly preferred is sodium dehydroacetate which can be purchased from Tri-K, as Tristat SDHA. Derivatives of dehydroacetic acid incude, but are not limited to, any compounds wherein the CH₃ groups are individually or in combination replaced by amides, esters, amino groups, alkyls, and alcohol esters. Tautomers of dehydroacetic acid are the isomers of dehydroacetic acid which can change into one another with great ease so that they ordinarily exist in equilibrium. Thus, tautomers of dehydroacetic acid can be described as having the chemical formula C₈H₈O₄ and generally having the structure above.

In one embodiment, the compositions of the present invention can comprise from about 0.001% to about 25% by weight of the composition, preferably from about 0.01% to about 10%, more preferably from about 0.05% to about 5%, and even more preferably from about 0.1% to about 1%, of dehydroacetic acid or pharmaceutically acceptable salts, derivatives or tautomers thereof.

24. Other Actives

The compositions of the present invention may contain a safe and effective amount of one or more of the following other actives or ingredients: fatty acids (especially poly-unsaturated fatty acids), glucosamine, zinc pyrithione (ZPT), anti-fungal agents, thiol compounds (e.g., N-acetyl cysteine, glutathione, thioglycolate), other vitamins (vitamin B 12), beta-carotene, ubiquinone, amino acids, and the like.

Dermatologically Acceptable Carrier

The topical compositions of the present invention also comprise a dermatologically acceptable carrier for the active materials. The phrase “dermatologically acceptable carrier”, as used herein, means that the carrier is suitable for topical application to the keratinous tissue, has good aesthetic properties, is compatible with the actives of the present invention and any other components, and will not cause any safety or toxicity concerns. A safe and effective amount of carrier is from about 50% to about 99.99%, preferably from about 60% to about 99.9%, more preferably from about 70% to about 98%, and even more preferably from about 80% to about 95% of the composition.

The carrier can be in a wide variety of forms. For example, emulsion carriers, including, but not limited to, oil-in-water, water-in-oil, silicone-in-water, water-in-silicone, water-in-oil-in-water, and oil-in-water-in-silicone emulsions, are useful herein.

Preferred carriers comprise an emulsion such as oil-in-water emulsions and water-in-oil emulsions, e.g., silicone-in-water or water-in-silicone emulsions. As will be understood by the skilled artisan, a given component will distribute primarily into either the water or oil phase, depending on the water solubility/dispensability of the component in the composition. Oil-in-water emulsions are especially preferred.

Emulsions according to the present invention generally contain a solution as described above and a lipid or oil. Lipids and oils may be derived from animals, plants, or petroleum and may be natural or synthetic (i.e., man-made). Preferred emulsions also contain a humectant, such as glycerin. Emulsions will preferably further contain from about 0.1% to about 10%, more preferably from about 0.2% to about 5%, of an emulsifier, based on the weight of the composition. Emulsifiers may be nonionic, anionic or cationic. Suitable emulsifiers are disclosed in, for example, U.S. Pat. No. 3,755,560, U.S. Pat. No. 4,421,769, and McCutcheon's Detergents and Emulsifiers, North American Edition, pages 317-324 (1986).

Suitable emulsions may have a wide range of viscosities, depending on the desired product form. Exemplary low viscosity emulsions, which are preferred, have a viscosity of about 50 centistokes or less, more preferably about 10 centistokes or less, even more preferably about 5 centistokes or less.

The compositions of the present invention can also comprise other dermatologically acceptable topical carriers and can also comprise oral carriers. For example, another topical carrier can be a surfactant-containing cleanser (e.g., bar, shampoo, foaming cleanser, liquid cleanser, body wash, cleansing cloth, and the like). In such a carrier, the surfactant can be anionic, cationic, zwitterionic, nonionic, or mixtures of these. Another topical carrier example is a color cosmetic (lipstick, rouge, eye liner, mascara, foundation, nail polish, and the like). An oral carrier can be a beverage, food item, pill, capsule, powder, caplet, and the like.

Optional Components

The compositions of the present invention may contain a variety of other ingredients that are conventionally used in given product types provided that they do not unacceptably alter the benefits of the invention.

The optional components, when incorporated into the composition, should be suitable for use in contact with human keratinous tissue without undue toxicity, incompatibility, instability, allergic response, and the like within the scope of sound judgment. The CTFA Cosmetic Ingredient Handbook, Second Edition (1992) describes a wide variety of nonlimiting cosmetic and pharmaceutical ingredients commonly used in the skin care industry, which are suitable for use in the compositions of the present invention. Examples of these ingredient classes include: abrasives, absorbents, aesthetic components such as fragrances, pigments, colorings/colorants, essential oils, skin sensates, astringents, etc. (e.g., clove oil, menthol, camphor, eucalyptus oil, eugenol, menthyl lactate, witch hazel distillate), anti-acne agents, anti-caking agents, antifoaming agents, antimicrobial agents (e.g., iodopropyl butylcarbamate), antioxidants, binders, biological additives, buffering agents, bulking agents, chelating agents, chemical additives, colorants, cosmetic astringents, cosmetic biocides, denaturants, drug astringents, external analgesics, film formers or materials, e.g., polymers, for aiding the film-forming properties and substantivity of the composition (e.g., copolymer of eicosene and vinyl pyrrolidone), opacifying agents, pH adjusters, propellants, reducing agents, sequestrants, skin bleaching and lightening agents, skin-conditioning agents, skin soothing and/or healing agents and derivatives, skin treating agents, thickeners, and vitamins and derivatives thereof.

Other optional components useful in the present invention include those described in U.S. Publication No. 2004-0175347A1, including desquamation actives, such as salicylic acid and zwitterionic surfactants; anti-acne actives, such as resorcinol, sulfur, erythromycin, zinc, dehydroacetic acid; anti-wrinkle actives/anti-atrophy actives; anti-oxidants/radical scavengers, such as tocopherol; chelators, such as furildioxime and derivatives thereof; flavonoids; anti-inflammatory agents; anti-cellulite agents; tanning actives such as dihydroxyacetone; skin lightening agents; antimicrobial and antifungal actives; sunscreen actives; conditioning agents such as glycerol, urea, petrolatum, sucrose polyester, and combinations thereof; thickening agents such as carboxylic acid polymers, crosslinked polyacrylate polymers, polyacrylamide polymers, polysaccharides, gums; water-soluble vitamins; and particulate materials.

### Composition Forms

The topical compositions of the subject invention, including but not limited to lotions, milks, mousses, serums, sprays, aerosols, foams, sticks, pencils, gels, creams and ointments, may comprise a dermatologically acceptable emollient. Such compositions preferably contain from about 2% to about 50% of the emollient. As used herein, “emollient” refers to a material useful for the prevention or relief of dryness, as well as for the protection of the skin. A wide variety of suitable emollients are known and may be used herein. Sagarin, Cosmetics, Science and Technology, 2nd Edition, Vol. 1, pp. 32-43 (1972), contains numerous examples of materials suitable as an emollient. A preferred emollient is glycerin. Glycerin is preferably used in an amount of from about 0.001 to about 20%, more preferably from about 0.01 to about 15%, and even more preferably from about 0.1 to about 10% by weight of the composition.

Compositions of this invention useful for cleansing (“cleansers”) are formulated with a suitable carrier (e.g., as described above, and from about 1% to about 90%, by weight of the composition, of a dermatologically acceptable surfactant).

The physical form of the cleansing compositions is not critical. The compositions can be, for example, formulated as toilet bars, liquids, shampoos, bath gels, hair conditioners, hair tonics, pastes, or mousses. Toilet bars are preferred since this is the form of cleansing agent most commonly used to wash the skin. Rinse-off cleansing compositions, such as shampoos, require a delivery system adequate to deposit sufficient levels of actives on the skin and scalp. A preferred delivery system involves the use of insoluble complexes. For a more complete disclosure of such delivery systems, see U.S. Pat. No. 4,835,148.

The compositions of the present invention may also be in the form of cosmetics. Suitable cosmetic forms include, but are not limited to, foundations, lipsticks, rouges, mascaras, and the like. Such cosmetic products may include conventional ingredients such as oils, colorants, pigments, emollients, fragrances, waxes, stabilizers, and the like. Exemplary carriers and such other ingredients which are suitable for use herein are described, for example, in U.S. Pat. No. 6,060,547.

The compositions of the present invention may also be in the form of shave prep products, including, for example, gels, foams, lotions, and creams; and include both aerosol and non-aerosol versions.

### Composition Preparation

The compositions of the present invention are generally prepared by conventional methods such as are known in the art of making topical compositions. Such methods typically involve mixing of the ingredients in one or more steps to a relatively uniform state, with or without heating, cooling, application of vacuum, and the like. The compositions are preferably prepared such as to optimize stability (physical stability, chemical stability, photostability) and/or delivery of the active materials. This optimization may include appropriate pH (e.g., less than 7), exclusion of materials that can complex with the active agent and thus negatively impact stability or delivery (e.g., exclusion of contaminating iron), use of approaches to prevent complex formation (e.g., appropriate dispersing agents or dual compartment packaging), use of appropriate photostability approaches (e.g., incorporation of sunscreen/sunblock, use of opaque packaging), etc.

### Methods for Regulating Keratinous Tissue Condition

The compositions of the present invention are useful for regulating a number of mammalian keratinous tissue conditions. Such regulation of keratinous tissue conditions includes prophylactic and therapeutic regulation. More specifically, such regulating methods are directed to, but are not limited to, thickening keratinous tissue (i.e., building the epidermis and/or dermis and/or subcutaneous layers of the skin and where applicable the keratinous layers of the nail and hair shaft); preventing, retarding, improving, and/or treating uneven skin tone by acting as a lightening or pigmentation reduction cosmetic agent; preventing, retarding, and/or treating atrophy of mammalian skin; softening and/or smoothing lips, hair and nails of a mammal; preventing, retarding, and/or treating itch of mammalian skin; preventing, retarding, and/or treating the appearance of dark under-eye circles and/or puffy eyes; preventing, retarding, and/or treating sallowness of mammalian skin; preventing, retarding, and/or treating sagging (i.e., glycation) of mammalian skin; preventing and/or retarding tanning of mammalian skin; desquamating, exfoliating, and/or increasing turnover in mammalian skin; reducing the size of pores in mammalian skin; regulating oily/shiny appearance of mammalian skin; preventing, retarding, and/or treating hyperpigmentation such as post-inflammatory hyperpigmentation; preventing, retarding, and/or treating the appearance of spider vessels and/or red blotchiness on mammalian skin; preventing, retarding, and/or treating fine lines and wrinkles of mammalian skin; preventing, retarding, and/or treating skin dryness (i.e., roughness, scaling, flaking) and preventing, retarding, and/or treating the appearance of cellulite in mammalian skin. The compositions of the present invention may also be useful in inhibiting hair growth, reducing shaving frequency, improving ease of shaving, decreasing shaving frequency, making hair softer and/or finer, making hair less noticeable, slowing the re-growth of hair, reducing erythema and/or irritation to skin, making skin smoother and/or silkier, and improving the hair removal process.

Regulating keratinous tissue condition involves topically applying to the keratinous tissue a safe and effective amount of a composition of the present invention. The amount of the composition that is applied, the frequency of application and the period of use will vary widely depending upon the level of skin and/or hair care actives and/or other components of a given composition and the level of regulation desired.

In a preferred embodiment, the composition is chronically applied to the skin. By “chronic topical application” is meant continued topical application of the composition over an extended period during the subject's lifetime, preferably for a period of at least about one week, more preferably for a period of at least about one month, even more preferably for at least about three months, even more preferably for at least about six months, and more preferably still for at least about one year. While benefits are obtainable after various maximum periods of use (e.g., five, ten or twenty years), it is preferred that chronic applications continue throughout the subject's lifetime. Typically applications would be on the order of about once per day over such extended periods, however application rates can vary from about once per week up to about three times per day or more.

A wide range of quantities of the compositions of the present invention can be employed to provide a skin appearance and/or feel benefit. Quantities of the present compositions, which are typically applied per application, are in mg composition/cm² skin, from about 0.1 mg/cm² to about 20 mg/cm². A particularly useful application amount is about 0.5 mg/cm² to about 10 mg/cm².

Regulating keratinous tissue condition is preferably practiced by applying a composition in the form of a skin lotion, clear lotion, milky lotion, cream, gel, foam, ointment, paste, emulsion, spray, conditioner, tonic, cosmetic, lipstick, foundation, nail polish, after-shave, or the like which is intended to be left on the skin or other keratinous tissue for some aesthetic, prophylactic, therapeutic or other benefit (i.e., a “leave-on” composition). After applying the composition to the keratinous tissue (e.g., skin), it is preferably left on for a period of at least about 15 minutes, more preferably at least about 30 minutes, even more preferably at least about 1 hour, even more preferably for at least several hours, e.g., up to about 12 hours. Any part of the external portion of the face, hair, and/or nails can be treated, e.g., face, lips, under-eye area, eyelids, scalp, neck, torso, arms, hands, legs, fingernails, toenails, scalp hair, eyelashes, eyebrows, etc. The application of the present compositions may be done using, e.g., the palms of the hands and/or fingers, an implement, e.g., a cotton ball, swab, wipe, pad, etc.

Another approach to ensure a continuous exposure of the keratinous tissue to at least a minimum level of the skin and/or hair care active is to apply the compound by use of a patch applied, e.g., to the face. Such an approach is particularly useful for problem skin areas needing more intensive treatment (e.g., facial crows feet area, frown lines, under eye area, and the like). The patch can be occlusive, semi-occlusive or non-occlusive. The composition can be contained within the patch or be applied to the skin prior to application of the patch. The patch can also include additional actives such as chemical initiators for exothermic reactions such as those described in PCT application WO 9701313. The patch can also contain a source of electrical energy (e.g., a battery) to, for example, increase delivery of the skin and/or hair care active and other active agents (e.g., iontophoresis). The patch is preferably left on the keratinous tissue for a period of at least about 5 minutes, more preferably at least about 15 minutes, more preferably still at least about 30 minutes, even more preferably at least about 1 hour, even more preferably at night as a form of night therapy.

Another approach to enhancing the benefits of the actives is use of a kit or regimen of 2 or 3 or 4 or more products and/or treatment procedures (e.g., exfoliation followed by topical treatment with one or more of the actives of the present invention, depilation of hair followed by topical treatment with one or more of the actives of the present invention, and the like). The various components of a regimen can be used in a short period of time (e.g., within an hour) or spread over a longer time frame within a day (e.g., morning and evening) or over even longer time periods (e.g., one step in the regimen done weekly or monthly and the other steps in the regimen done on a more regular basis, e.g., daily). A kit or regimen can also consist of combinations of the carriers and/or product forms noted above, e.g., two or more of a cleanser, a topical leave-on treatment, and an oral supplement.

The present invention also contemplates the delivery of energy, via a device, to keratinous tissue, either simultaneously and/or sequentially with application of the topical compositions. The energy delivery device may deliver energy in a variety of forms, including but not limited to energy in the form of light, heat, sound (including ultrasonic waves), magnetic energy, electromagnetic energy (including radiofrequency waves and microwaves), and combinations thereof. The delivery of energy may be continuous, pulsed, modulated, non-modulated, and combinations thereof. In one embodiment, the energy delivery device is hand-held. Alternatively, the energy delivery device is cordless.

The energy may be applied by holding a device within a single area of keratinous tissue, and subsequently moving the device to another area of tissue (or “stamping”). Alternatively, the energy may be applied as the device is continuously moved, or scanned, across the surface of the tissue. The device may be held in substantially continuous contact with the surface of the keratinous tissue, as with laser devices, or may be held at a short distance from the keratinous tissue with the energy directed toward the surface, as with flash lamps.

A temperature change may be simultaneously induced in the keratinous tissue or alternatively, in a compound applied to the surface of the tissue. This temperature change is in addition to any temperature change induced by the delivered energy itself. For example, the keratinous tissue may be slightly warmed prior to delivery of energy, or alternatively, the keratinous tissue may be cooled after delivery of energy.

For energy derived from ultraviolet light sources, the wavelength will generally fall within the UV-A range, from about 315-400 nm, where “nm” means 1×10-9 meters. For energy derived from visible light sources, the wavelength will generally range from about 400 nm to about 700 nm. For energy derived from infrared (IR) light sources, the wavelength will generally range from about 700 nm to about to about 3000 run. The amount of energy delivered, or “output fluence,” may be in the range of about 1 J/cm2 to about 100 J/cm2, where “J” means Joules. For pulsed light sources, the pulse length may range from about 0.001 seconds to about 3 seconds, with an average pulse duration of from about 0.001 seconds to about 1 second. The surface area of keratinous tissue to be covered will vary depending on the application. These and other parameters relevant to delivery of energy depend upon the type of treatment and the type of tissue to be treated, and will appropriately be selected by one of skill in the art.

### Articles of Commerce

The present invention also provides articles of commerce that include personal care compositions and indicia (and/or an image) related to at least one of packaging for the personal care composition and advertisement material pertaining to the personal care composition which communicates that topical application of the personal care compostion may improve one's skin tone or skin color.

In one exemplary embodiment, packaging for the personal care compsoiton comprises the indicia and/or image. The packaging may be primary packaging, secondary packaging, and/or additional packaging. The type of packaging associated with the present invention is unlimited. The packaging can be made from a variety of materials, can be made in numerous configurations, and can be made with any manufacturing techniques known to the skilled artisan. Exemplary packaging embodiments include boxes, bags, pouches, paperboard cans, bottles, tottles, jars, thermoform blisters, clamshells, and combinations thereof. Other packaging embodiments are equally suitable.

In another exemplary embodiment, advertisement materials and/or devices pertaining to the personal care composition comprises the indicia and/or image.

Exemplary advertisement materials/devices include, but are not limited to, point-of-sale devices and/or materials, sample products and related information, coupons, mailers, periodical advertisement documents, product brochures, product inserts, product displays, shelf talkers, billboards, posters, buses, outdoor seating, and any other advertisement media available to prospective buyers. Advertisement materials/devices can be affixed (permanently or removably) to packaging, or included within the packaging, for example.

### EXAMPLES

The following are non-limiting examples of the compositions of the present invention. The examples are given solely for the purpose of illustration and are not to be construed as limitations of the present invention, as many variations thereof are possible without departing from the spirit and scope of the invention, which would be recognized by one of ordinary skill in the art. In the examples, all concentrations are listed as weight percent, unless otherwise specified and may exclude minor materials such as diluents, filler, and so forth. The listed formulations, therefore, comprise the listed components and any minor materials associated with such components. As is apparent to one of ordinary skill in the art, the selection of these minors will vary depending on the physical and chemical characteristics of the particular ingredients selected to make the present invention as described herein.

Content in formulation (g component per 100 g formulation)

| Component | A | B | C | D | E | F |
|---|---|---|---|---|---|---|
| Disodium EDTA | 0.100 | 0.100 | 0.100 | 0.100 | 0.100 | 0.100 |
| Carnosine | 5.000 | 0 | 0 | 0 | 0 | 0 |
| Vanillin acetate | 0 | 2.000 | 0 | 0 | 0 | 0 |
| Tetrahydrocurcumin | 0.500 | 0.500 | 0.500 | 1.000 | 0.250 | 0.500 |
| Glycyrrhetinic acid | 0 | 0 | 0 | 0.300 | 0 | 0 |
| Thiotaine ® | 0 | 0 | 0 | 0 | 5.000 | 0 |
| Sinablanca ® | 0 | 0 | 0 | 0 | 0 | 2.500 |
| Niacinamide | 4.000 | 4.000 | 4.000 | 4.000 | 4.000 | 4.000 |
| Citric acid | 1.500 | 0 | 0 | 0 | 0 | 0 |
| Isohexadecane | 3.000 | 3.000 | 0 | 0 | 3.000 | 3.000 |
| Isopropyl isostearate | 1.330 | 1.330 | 0 | 0 | 1.330 | 1.330 |
| Isopropyl N-laurosylsarcosinate | 0 | 0 | 5.000 | 6.000 | 0 | 0 |
| Sucrose polycottonseedate | 0.670 | 0.670 | 0 | 0 | 0.670 | 0.670 |
| Polymethylsilsesquioxane | 0.250 | 0.250 | 0.250 | 0.250 | 0.250 | 0.250 |
| Cetearyl glucoside + cetearyl alcohol | 0.200 | 0.200 | 0.200 | 0.200 | 0.200 | 0.200 |
| Behenyl alcohol | 0.400 | 0.400 | 0.400 | 0.400 | 0.400 | 0.400 |
| Ethylparaben | 0.200 | 0.200 | 0.200 | 0.200 | 0.200 | 0.200 |
| Propylparaben | 0.100 | 0.100 | 0.100 | 0.100 | 0.100 | 0.100 |
| Cetyl alcohol | 0.320 | 0.320 | 0.320 | 0.320 | 0.320 | 0.320 |
| Stearyl alcohol | 0.480 | 0.480 | 0.480 | 0.480 | 0.480 | 0.480 |
| Tocopheryl acetate | 0.500 | 0.500 | 0.500 | 0.500 | 0.500 | 0.500 |
| PEG-100 stearate | 0.100 | 0.100 | 0.100 | 0.100 | 0.100 | 0.100 |
| Glycerin | 7.000 | 7.000 | 7.000 | 7.000 | 7.000 | 7.000 |
| Titanium dioxide | 0.604 | 0.604 | 0.604 | 0.604 | 0.604 | 0.604 |
| Polyacrylamide + C13-14 isoparaffin + laureth-7 | 3.000 | 2.000 | 2.000 | 2.000 | 2.000 | 2.000 |
| Panthenol | 1.000 | 1.000 | 1.000 | 1.000 | 1.000 | 1.000 |
| Benzyl alcohol | 0.400 | 0.400 | 0.400 | 0.400 | 0.400 | 0.400 |
| Dimethicone + dimethiconol | 2.000 | 2.000 | 2.000 | 2.000 | 2.000 | 2.000 |
| Water (to 100 g) | to | to | to | to | to | to |
| | 100 | 100 | 100 | 100 | 100 | 100 |
| TOTAL | 100 | 100 | 100 | 100 | 100 | 100 |

Content in formulation (g component per 100 g formulation)

| Component | G | H | I | J | K | L |
|---|---|---|---|---|---|---|
| Disodium EDTA | 0.100 | 0.100 | 0.100 | 0.100 | 0.100 | 0.100 |
| Glucosyl hesperidin | 1.000 | 0 | 0 | 0 | 0 | 0 |
| Glycyrrhizic acid, ammonium salt | 0 | 0.300 | 0 | 0 | 0 | 0 |
| Cetyl pyridinium chloride | 0 | 0 | 0.200 | 0 | 0 | 0 |
| Menthyl anthranilate | 0 | 0 | 0 | 5.000 | 0 | 0 |
| Oxynex ® | 0 | 0 | 0 | 0 | 2.000 | 0 |
| Butylated hydroxyl toluene | 0 | 0 | 0 | 0 | 0 | 1.000 |
| Tetrahydrocurcumin | 0.500 | 0.500 | 0.500 | 1.000 | 0.500 | 1.000 |
| Niacinamide | 4.000 | 4.000 | 4.000 | 4.000 | 4.000 | 4.000 |
| Polyquaternium 37 | 0 | 0 | 1.500 | 0 | 0 | 0 |
| Isohexadecane | 3.000 | 3.000 | 3.000 | 3.000 | 3.000 | 3.000 |
| Isopropyl isostearate | 1.330 | 1.330 | 1.330 | 1.330 | 1.330 | 1.330 |
| Sucrose polycottonseedate | 0.670 | 0.670 | 0.670 | 0.670 | 0.670 | 0.670 |
| Polymethylsilsesquioxane | 0.250 | 0.250 | 0.250 | 0.250 | 0.250 | 0.250 |
| Cetearyl glucoside + cetearyl alcohol | 0.200 | 0.200 | 0.200 | 0.200 | 0.200 | 0.200 |
| Behenyl alcohol | 0.400 | 0.400 | 0.400 | 0.400 | 0.400 | 0.400 |
| Ethylparaben | 0.200 | 0.200 | 0.200 | 0.200 | 0.200 | 0.200 |
| Propylparaben | 0.100 | 0.100 | 0.100 | 0.100 | 0.100 | 0.100 |
| Cetyl alcohol | 0.320 | 0.320 | 0.320 | 0.320 | 0.320 | 0.320 |
| Stearyl alcohol | 0.480 | 0.480 | 0.480 | 0.480 | 0.480 | 0.480 |
| Tocopheryl acetate | 0.500 | 0.500 | 0.500 | 0.500 | 0.500 | 0.500 |
| PEG-100 stearate | 0.100 | 0.100 | 0.100 | 0.100 | 0.100 | 0.100 |
| Glycerin | 7.000 | 7.000 | 7.000 | 7.000 | 7.000 | 7.000 |
| Titanium dioxide | 0.604 | 0.604 | 0.604 | 0.604 | 0.604 | 0.604 |
| Polyacrylamide + C13-14 isoparaffin + laureth-7 | 2.000 | 2.000 | 0 | 2.000 | 2.000 | 2.000 |
| Panthenol | 1.000 | 1.000 | 1.000 | 1.000 | 1.000 | 1.000 |
| Benzyl alcohol | 0.400 | 0.400 | 0.400 | 0.400 | 0.400 | 0.400 |
| Dimethicone + dimethiconol | 2.000 | 2.000 | 2.000 | 2.000 | 2.000 | 2.000 |
| Water (to 100 g) | to | to | to | to | to | to |
| | 100 | 100 | 100 | 100 | 100 | 100 |
| TOTAL | 100 | 100 | 100 | 100 | 100 | 100 |

| Component | M | N | O | P |
|---|---|---|---|---|
| Disodium EDTA | 0.1 | 0.1 | 0.1 | 0.1 |
| Niacinamide | 4.0 | 4.0 | 4.0 | 0 |
| Tetrahydrocurcumin | 0.5 | 1.0 | 1.0 | 0.5 |
| Hexamidine diisethionate | 0.1 | 1.0 | 1.0 | 0.1 |
| N-undecylenoyl-L-phenylalanine | 0.0 | 0.0 | 0.2 | 0 |
| Sodium dehydroacetate | 0.0 | 0.0 | 0.2 | 0.5 |
| Isohexadecane | 0 | 3.0 | 3.0 | 3.0 |
| Isopropyl isostearate | 0 | 1.33 | 1.33 | 1.33 |
| Isopropyl N-laurosylsarcosinate | 6.0 | 0 | 0 | 0 |
| Sucrose polycottonseedate | 0.67 | 0.67 | 0.67 | 0.67 |
| Polymethylsilsesquioxane | 0.25 | 0.25 | 0.25 | 0.25 |
| Cetearyl glucoside + cetearyl | 0.2 | 0.2 | 0.2 | 0.2 |
| alcohol | | | | |
| Behenyl alcohol | 0.4 | 0.4 | 0.4 | 0.4 |
| Ethylparaben | 0.2 | 0.2 | 0.2 | 0.2 |
| Propylparaben | 0.1 | 0.1 | 0.1 | 0.1 |
| Cetyl alcohol | 0.32 | 0.32 | 0.32 | 0.32 |
| Stearyl alcohol | 0.48 | 0.48 | 0.48 | 0.48 |
| Tocopheryl acetate | 0.5 | 0.5 | 0.5 | 0.5 |
| PEG-100 stearate | 0.1 | 0.1 | 0.1 | 0.1 |
| Glycerin | 7.0 | 7.0 | 7.0 | 7.0 |
| Titanium dioxide | 0.604 | 0.604 | 0.604 | 0.604 |
| Polyacrylamide + C13-14 | 2.0 | 2.0 | 2.0 | 2.0 |
| isoparaffin + laureth-7 | | | | |
| Panthenol | 1.0 | 1.0 | 1.0 | 1.0 |
| Benzyl alcohol | 0.4 | 0.4 | 0.4 | 0.4 |
| Dimethicone + dimethiconol | 2.0 | 2.0 | 2.0 | 2.0 |
| Water (to 100 g) | to 100 | to 100 | to 100 | to 100 |
| TOTAL | 100 | 100 | 100 | 100 |

| Component | Q | R |
|---|---|---|
| Disodium EDTA | 0.100 | 0.100 |
| Hydroquinone | 2.000 | 0 |
| Tetrahydrocurcumin | 0.500 | 1.000 |
| Retyinyl propionate | 0 | 0.300 |
| Niacinamide | 4.000 | 4.000 |
| Isohexadecane | 3.000 | 3.000 |
| Isopropyl isostearate | 1.330 | 1.330 |
| Sucrose polycottonseedate | 0.670 | 0.670 |
| Polymethylsilsesquioxane | 0.250 | 0.250 |
| Cetearyl glucoside + cetearyl alcohol | 0.200 | 0.200 |
| Behenyl alcohol | 0.400 | 0.400 |
| Ethylparaben | 0.200 | 0.200 |
| Propylparaben | 0.100 | 0.100 |
| Cetyl alcohol | 0.320 | 0.320 |
| Stearyl alcohol | 0.480 | 0.480 |
| Tocopheryl acetate | 0.500 | 0.500 |
| PEG-100 stearate | 0.100 | 0.100 |
| Glycerin | 7.000 | 7.000 |
| Titanium dioxide | 0.604 | 0.604 |
| Polyacrylamide + C13-14 isoparaffin + laureth-7 | 2.000 | 2.000 |
| Panthenol | 1.000 | 1.000 |
| Benzyl alcohol | 0.400 | 0.400 |
| Dimethicone + dimethiconol | 2.000 | 2.000 |
| Water (to 100 g) | to | to |
| | 100 | 100 |

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

All documents cited in the Background, Summary of the Invention, and Detailed Description of the Invention are, in relevant part, incorporated herein by reference; the citation of any document is not to be construed as an admission that it is prior art with respect to the present invention.

What is claimed is:

1. A personal care composition comprising:
a) tetrahydrocurcumin; and
b) at least one additional skin and/or hair care active selected from the group consisting of sugar amine, vitamin B₃, retinoids, hydroquinone, peptides, phytosterol, dialkanoyl hydroxyproline, hexamidine, salicylic acid, n-acyl amino acid compounds, sunscreen actives, water soluble vitamins, oil soluble vitamins, hesperedin, mustard seed extract, glycyrrhizic acid, glycyrrhetinic acid, carnosine, Butylated Hydroxytoluene (BHT) and Butylated Hydroxyanisole (BHA), menthyl anthranilate, cetyl pyridinium chloride, ergothioneine, vanillin or its derivatives, diethylhexyl syrinylidene malonate, melanostatine, sterol esters, idebenone, dehydroacetic acid, yeast extract, beta glucans, alpha glucans, their salts, their derivatives, their precursors, and/or combinations thereof, and
c) a dermatologically acceptable carrier.

2. The personal care composition of claim 1, wherein the tetrahydrocurcumin is used in combination with tetrahydrodemethoxycurcumin.

3. The personal care composition of claim 1, wherein the tetrahydrocurcumin is used in combination with tetrahydrobisdemethoxycurcumin.

4. The personal care composition of claim 1, wherein the tetrahydrocurcumin is used in combination with both tetrahydrodemethoxycurcumin and tetrahydrobisdemethoxycurcumin

5. The personal care composition of claim 1, wherein the at least one additional skin and/or hair care active is vitamin B₃.

6. The personal care composition of claim 1, wherein the at least one additional skin and/or hair care active comprises niacinamide, panthenol, and vitamin E acetate.

7. The personal care composition of claim 1, wherein the at least one additional skin and/or hair care active comprises a yeast extract and ascorbyl glucoside.

8. The personal care composition of claim 1, further comprising from about 0.001% to about 10%, by weight, of an additional component selected from the group consisting of desquamatory actives, anti-acne actives, wrinkle repair actives, anti-oxidants, radical scavengers, chelators, flavonoids, anti-inflammatory agents, anti-cellulite agents, skin lightening agents, antimicrobial actives, antifungal actives, conditioning agents, thickening agents, water soluble vitamins, oil soluble vitamins, particulate material, topical anesthetics, and combinations thereof.

9. A personal care composition comprising at least one anti-oxidant compound, at least one tyrosinase inhibiting compound, at least one trypsin inhibiting compound, at least one anti-inflammatory compound, and at least one nitric oxide scavenging compound.

10. An article of commerce, comprising:
a) a personal care composition comprising tetrahydrocurcumin and at least one sunscreen active; and
b) at least one of the personal care composition, packaging for the personal care composition and advertisement material pertaining to the personal care composition comprising indicia and/or an image which communicates that topical application of the personal care compostion may improve one's skin tone or skin color.

11. The article of commerce according to claim 10, wherein the personal care composition further comprises tetrahydromethoxycurcumin.

12. The article of commerce according to claim 10, wherein the personal care composition further comprises tetrahydrobisdemthoxycurcumin.

13. The article of commerce according to claim 11, wherein the personal care composition further comprises tetrahydrobisdemthoxycurcumin.

14. The article of commerce according to claim 10, wherein the indica and/or imiage further communicates that the personal care composition provides UV protection.

15. The article of commerce according to claim 10, wherein the personal care compostion further comprises vitamin B and vitamin E.

16. The article of commerce according to claim 15, wherein the vitamin B comprises niacinamide and panthenol, and wherine the vitamin E comprises vitamin E acetate.

17. The article of commerce according to claim 10, wherein the personal care composition further comprises a yeast extract.

21. The article of commerce according to claim 17, wherein the personal care composition further comprises ascorbyl glucoside.

22. A method of regulating the condition of mammalian keratinous tissue, the method comprising the step of topically applying to the skin of a mammal in need of such treatment the composition of claim 1.

23. A method of improving skin tone or skin color, the method comprising the step of topically applying to the skin of a mammal in need of such treatment the composition of claim 1.

24. A method of skin lightening, the method comprising the step of topically applying to the skin of a mammal in need of such treatment the composition of claim 1.

25. A method of inhibiting hair growth, the method comprising the step of topically applying to the skin of a mammal in need of such treatment the composition of claim 1.

26. A method of regulating the condition of mammalian keratinous tissue, the method comprising the step of topically applying to the skin of a mammal in need of such treatment the composition of claim 9.

27. A method of improving skin tone or skin color, the method comprising the step of topically applying to the skin of a mammal in need of such treatment the composition of claim 9.

28. A method of skin lightening, the method comprising the step of topically applying to the skin of a mammal in need of such treatment the composition of claim 9.
